(19) United States
(12) Patent Application Publication
(10) Pub. No.: US 2020/0383763 A1
(43) Pub. Date: Dec. 10, 2020

(54) INSULATED EQUINE THERAPY BOOT

(71) Applicants: Miranda LUDOVICE, Raleigh, NC (US); Jerry LIN, Arlington, VA (US); Emily SOLON, Canton, GA (US); Yuyan WANG, Doral, FL (US); UNIVERSITY OF GEORGIA RESEARCH FOUNDATION, INC., Athens, GA (US)

(72) Inventors: Miranda LUDOVICE, Raleigh, NC (US); Jerry LIN, Arlington, VA (US); Emily SOLON, Canton, GA (US); Yuyan WANG, Doral, FL (US); Andrew H PARKS, Athens, GA (US); Jarred M. WILLIAMS, Athens, GA (US)

(21) Appl. No.: 16/607,153

(22) PCT Filed: Apr. 24, 2018
(86) PCT No.: PCT/US18/29081

§ 371 Date: Oct. 22, 2019

Related U.S. Application Data
(60) Provisional application No. 62/489,193, filed on Apr. 24, 2017.

(51) Int. Cl. A61D 9/00; A01L 15/00; A01L 7/02; A01K 13/00; A61F 7/10

(52) U.S. Cl. CPC A61D 9/00; A01L 15/00; A61F 2007/0043; A01K 13/007; A61F 7/10; A01L 7/02

(57) ABSTRACT

An equine therapy boot that has an insulating shaft that can be securely fastened at multiple locations on an equine leg and hold a volume of therapeutic media to treat the equine hoof and leg is disclosed herein. The equine therapy boot includes a sole member, an internal securing mechanism, and an insulating shaft configured to retain therapeutic media. Advantageously, this equine therapy boot secures the horse's hoof to the sole member such that the insulating shaft is not displaced from the horse's hoof and leg during the treatment period. Also disclosed herein are methods of treating a diseased or injured horse's hoof and leg with the disclosed equine therapy boot.

### CROSS REFERENCE TO RELATED APPLICATION

The subject matter described in this application is related to subject matter disclosed in the U.S. Patent Provisional App. No. 62/489,193 filed Apr. 24, 2017, entitled “Insulated Equine Therapy Boot,” which is incorporated herein by reference in its entirety.

### BACKGROUND

Horses are ungulates. This means horses have one digit on each leg to support their body weight. As a result, any injury to the leg or hoof of a horse can cause serious discomfort or fatality. Additionally, since horses primarily stand, treating an injured hoof or leg can be a lengthy and difficult process. Equine boots or similar contraptions are often used to treat horses suffering from hoof or leg diseases or injury. When an equine boot is used to treat a horse's hoof or leg, it is especially necessary for the equine boot to remain in place on the horse's leg to maximize treatment therapy. This is especially the case when a horse is suffering from a disease like laminitis where the horse's hoof and leg are often treated with cryotherapy (cold therapy) for prolonged periods of time.

Cryotherapy is one of the most effective treatments for equine hoof and leg disease or injury. Current cryotherapy methods include applying an ice sleeve to the horse's limb, such as the ice sleeves depicted in FIGS. 1 and 2, or even restraining a horse in a pool of cold water or placing the horse's leg in a bucket or container of cold water for an extended period of time. The cryotherapy treatment illustrated in FIGS. 1 and 2 is inefficient because, for example, the force of the horse's hoof and leg as it moves punctures or tears the ice sleeve causing the ice sleeve to leak. Additionally, sweat on the external surface of the ice sleeve forms as the ice melts, causing the tape to loosen, resulting in the horse walking out of the ice sleeve or causing the ice sleeve to become detached from the horse's hoof and leg. These current methods do not provide effective cryotherapy treatment for more than two hours before a caregiver must remove the ice sleeves and re-administer the cryotherapy treatment. As a result, these current methods are labor intensive and require frequent monitoring of the horse which makes cryotherapy a prohibitive form of treatment that is difficult to perform outside of a veterinary hospital. Equine ice boots have been developed to attempt to address the shortcomings resulting from these treatment methods. However, these conventional ice boots fail to adequately secure to the horse's leg resulting in the horse walking out of the ice boot or otherwise displacing its hoof and leg in the ice boot such that treatment is ineffective.

### SUMMARY

The equine therapy boot disclosed herein includes an internal securing mechanism configured to secure the horse's hoof flat to the sole member of the therapy boot such that the horse's leg maintains a secured position within the therapy boot. The internal securing mechanism prevents the horse from walking out of or otherwise displacing its hoof and leg in the therapy boot thus reducing the need to monitor the horse or consistently replace the detached therapy boot. As a result, the disclosed equine therapy boot provides for a more effective and less labor-intensive treatment period.

This equine therapy boot includes a sole member having a bottom side for contacting the ground and a top side for contact with at least a portion of the sole of a horse's hoof, an internal securing mechanism for securing the horse's leg within the boot such that the portion of the sole of the horse's hoof is in contact with the top side of the sole member, the internal securing mechanism having a first plurality of straps for attaching to a portion of the horse's leg proximal to the horse's hoof, and an insulating shaft wherein the insulating shaft extends upward from the sole member wherein the insulating shaft is configured to retain media.

In some embodiments, the insulating shaft is secured to the horse's leg by a second plurality of straps at a point proximal to the first plurality of straps. In some embodiments, the first plurality of straps further includes a first garter strap configured to extend around the horse's pastern and at least one suspending strap that extends from the sole member to the first garter strap. In some embodiments, the second plurality of straps includes a second garter strap configured to extend around the horse's leg and at least one suspending strap that extends from the insulating shaft to the second garter strap.

In some embodiments, the insulating shaft is attached to the sole member. In some embodiments, the distal end of the insulating shaft is closed and the sole member is disposed within the insulating shaft such that the bottom side of the sole member is attached to the closed distal end of the insulating shaft.

In some embodiments, the insulating shaft contains at least one zipper extending from a bottom of the insulating shaft to a top of the insulating shaft. In some embodiments, the at least one zipper is a two-way zipper.

In some embodiments, the insulating shaft further includes an external layer, an insulating layer, and an internal layer. The external layer, the insulating layer, and the internal layer form a reservoir for retaining media, such as a medical soak or a combination of ice and water. In some embodiments, the internal layer is attached to the external layer. In some embodiments, the internal layer is a waterproof material. In some embodiments, the insulating layer is an insulating material that reduces the transmission of heat. In some embodiments, the external layer is a protective material. In some embodiments, a mesh panel is attached to the internal layer.

In some embodiments, the insulating shaft further includes a top portion and the top portion substantially tapers around the horse's leg. In some embodiments, the sole member is a cushioning pad.

Methods for treating a diseased or injured horse's hoof and leg are disclosed herein. Methods of treatment include the step of providing an equine therapy boot, the equine therapy boot comprising a sole member having a bottom side for contacting the ground and a top side for contacting at least a portion of the sole of the horse's hoof, an internal securing mechanism for securing the horse's leg within the boot such that the portion of the sole of the horse's hoof is in contact with the top side of the sole member, the internal securing mechanism further comprising a first plurality of straps for attaching to a portion of the horse's leg proximal to the horse's hoof, and an insulating shaft wherein the insulating shaft extends upwards from the sole member wherein the insulating shaft is configured to retain media. The method further includes fitting the equine therapy boot to the horse. In some methods, the method includes administering media to the insulating shaft of the equine therapy boot. In some methods, the method includes maintaining the equine therapy boot in its position with the at least a portion of the sole of the horse's hoof in contact with the top side of the sole member for a period of time.

The method of treatment further includes maintaining the equine therapy boot in its position with the at least a portion of the sole of the horse's hoof in contact with the top side of the sole member for a period of time greater than 2 hours. The method can further include maintaining the equine therapy boot in its position with the at least a portion of the sole of the horse's hoof in contact with the top side of the sole member for a period of time of 4, 6, 8, 10, 12, 24, 48, 72 hours, or therebetween.

In some treatment methods, the administered media is cooler than the temperature of the horse's hoof. In other treatment methods, the administered media is warmer than the temperature of the horse's hoof.

### BRIEF DESCRIPTION OF THE DRAWINGS

Example features and embodiments are disclosed in the accompanying drawings. However, the present disclosure is not limited to the precise arrangements and instrumentalities shown.

FIG. 1 is a perspective view of a prior art ice sleeve.

FIG. 2 is a perspective view of a prior art ice sleeve.

FIG. 3 is a front perspective view of one embodiment of the equine therapy boot.

FIG. 4 is a front perspective view of one embodiment of the internal securing mechanism.

FIG. 5 is a side perspective view of the internal securing mechanism shown in FIG. 4.

FIG. 6 is a perspective view of one embodiment of the equine therapy boot.

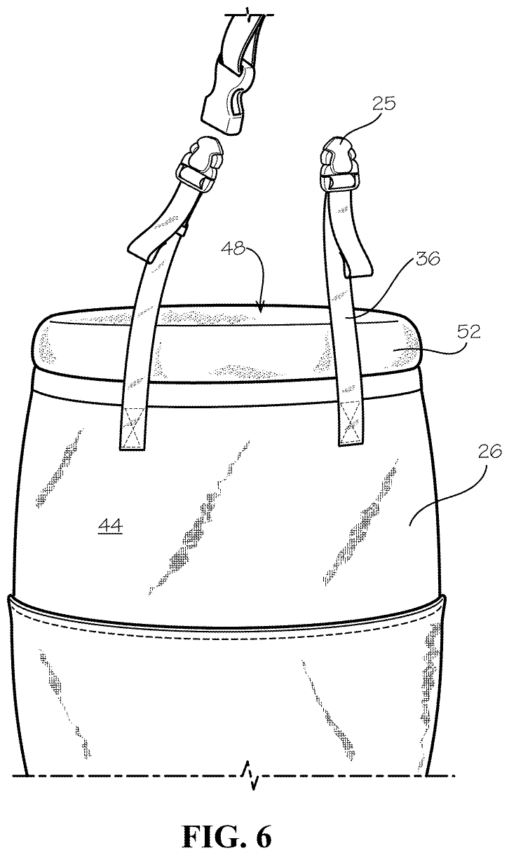

FIG. 7 is a perspective view of the internal surface of one embodiment of the insulating shaft.

FIG. 8 is a perspective view of a horse's leg secured in one embodiment of the equine therapy boot.

FIG. 9 is a side perspective view of one embodiment of the equine therapy boot.

### DETAILED DESCRIPTION

Certain terminology is used herein for convenience only and is not to be taken as a limitation on the present invention. In the drawings, the same reference numbers are employed for designating the same elements throughout the several figures. A number of examples are provided, nevertheless, it will be understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, the terms “distal” and “proximal” are anatomical terms used to describe the position of the equine therapy boot relative to the horse's topline. “Distal” is farther from the topline, whereas “proximal” is closer to the topline.

As used herein, “attached” or “attaching” is defined as securing two edges in a permanent fashion via sewing, gluing, bonding, or another similar type of attachment method known in the art.

The following is a description of various embodiments of an equine therapy boot that utilizes an internal securing mechanism to secure the therapy boot to the horse's hoof and leg such that the boot is not displaced during the treatment period.

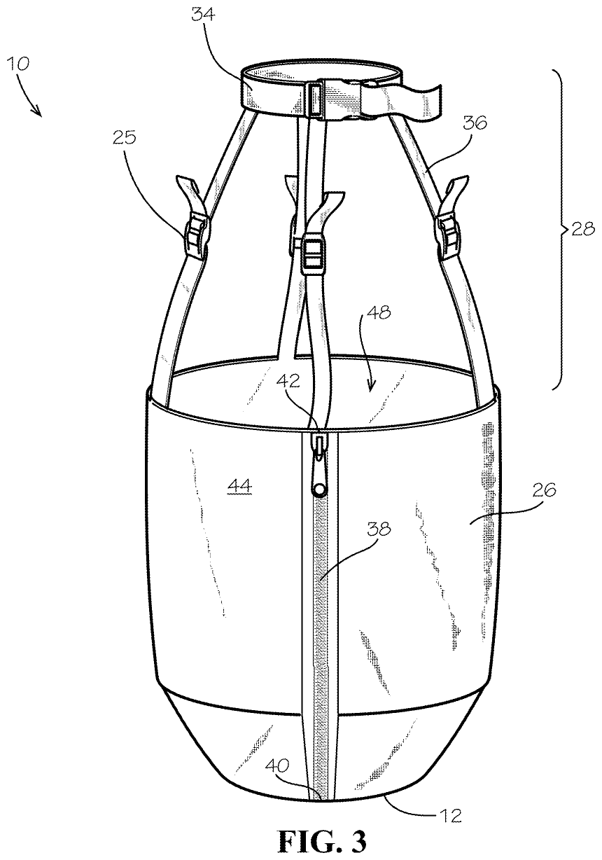

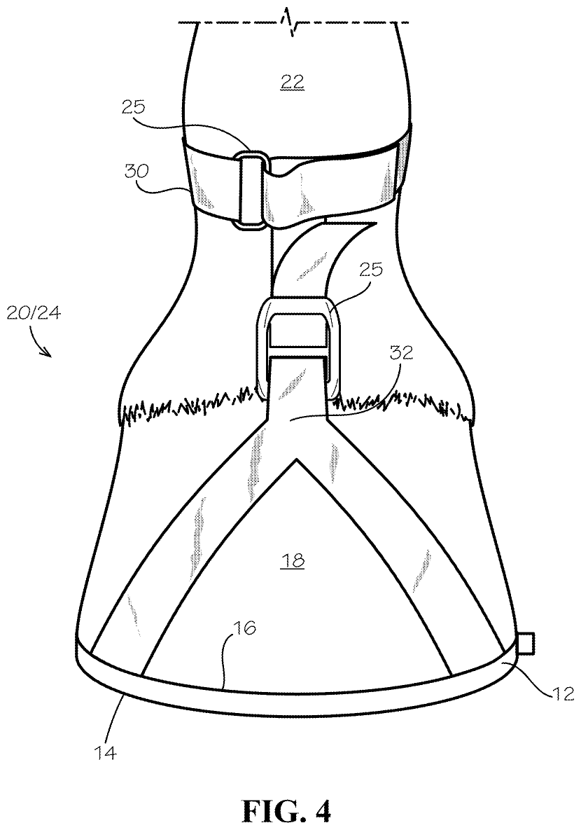

The equine therapy boot 10 of FIGS. 3 and 4 include a sole member 12 having a bottom side 14 for contacting the ground and a top side 16 for contact with at least a portion of the sole of a horse's hoof 18. The sole member 12 may comprise any durable material that is designed to withstand the force of horse's leg, such as, rubber, polyurethane, or polyvinyl chloride (PVC). In some embodiments, the sole member 12 is a cushioning pad that is designed to absorb concussion and dissipate force. The equine therapy boot 10 also includes an internal securing mechanism 20 for securing the horse's leg 22 within the boot 10 such that the portion of the sole of the horse's hoof 18 is in contact with, and rests substantially on, the top side 16 of the sole member 12. The internal securing mechanism 20 holds the horse's hoof 18 flat to the sole member 12 such that when the horse lifts its leg to walk or otherwise move, ice or other solids are prohibited from getting wedged between the sole of the horse's hoof 18 and the sole member 12.

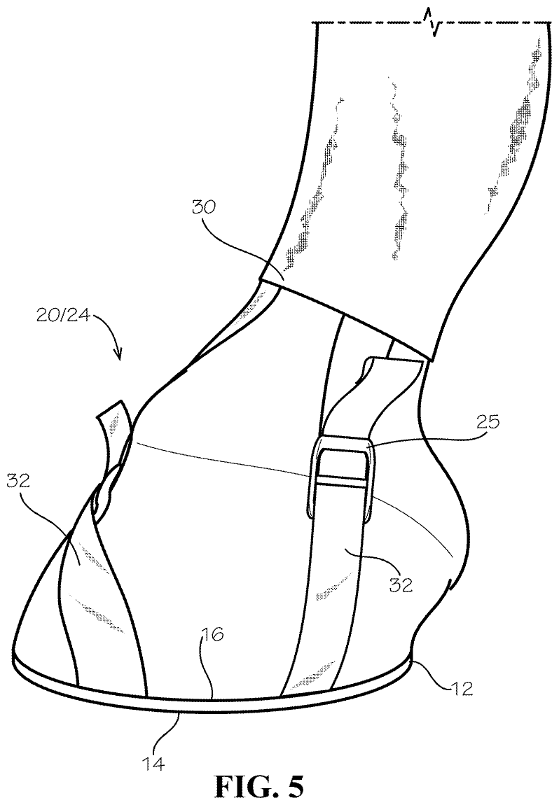

As depicted in FIGS. 4 and 5, the internal securing mechanism 20 has a first plurality of straps 24 for attaching to a portion of the horse's leg 22 proximal to the horse's hoof 18. In some embodiments, the first plurality of straps 24 includes a first garter strap 30 configured to extend around the horse's pastern and at least one suspending strap 32 that extends from the sole member 12 to the first garter strap 30. In some embodiments, such as the embodiment depicted in FIG. 4, the at least one suspending strap 32 has two points of contact with the sole member 12, such that the at least one suspending strap 32 forms a sandal-like strap to secure the horse's hoof 18 in the equine therapy boot 10. In some embodiments, the first plurality of straps 24 are adjustable such that the equine therapy boot 10 may be adjusted to fit horses of varying sizes. In some embodiments the first plurality of straps 24 are made of flexible material such as nylon, leather, or neoprene, and can further include removable fasteners 25 such as hook and loop tape, buckles, snaps, Velcro, or other forms of fasteners known in the art. In some embodiments, the removeable fasteners 25 secure the first plurality of straps 24 around the horse's hoof and pastern region such that the internal securing mechanism 20 can be fitted to the horse's hoof 18 and leg 22.

The equine therapy boot 10 further includes an insulating shaft 26. The insulating shaft 26 extends upward from the sole member 12. The insulating shaft 26 is configured to retain media for treating a horse's diseased or injured hoof 18 and leg 22. This media may include heated water, cold water, a combination of ice and water, medical soaks, medicated wraps, protecting politics, ice packs, hot packs, topical treatments, or other treatment medias.

In some embodiments, the insulating shaft 26 is secured to the horse's leg 22 by a second plurality of straps 28 at a point proximal to the first plurality of straps 24. In some embodiments, the second plurality of straps 28 are adjustable such that the equine therapy boot 10 may be fitted to horses of varying sizes. In some embodiments, such as the embodiment depicted in FIG. 3, the second plurality of straps 28 includes a second garter strap 34 configured to extend around the horse's leg 22 and at least one suspending strap 36 that extends from the insulating shaft 26 to the second garter strap 34. In some embodiments, the second garter strap 34 is proximal to the horse's carpus or hock. In some embodiments, the second plurality of straps 28 are made of a flexible material such as nylon, leather, or neoprene, and can further include removable fasteners 25 such as hook and loop tape, buckles, snaps, Velcro, or other forms of fasteners known in the art. In some embodiments, the at least one suspending strap 36 contains a removable fastener 25 to vertically adjust the strap length to allow for different proportions or heights of the horse's leg. In some embodiments, the removeable fasteners secure the second garter strap 34 around the horse's leg such that the equine therapy boot 10 stays in place during the treatment period without slipping off.

In some embodiments, the insulating shaft 26 is attached to the sole member 12. In some embodiments, the distal end of the insulating shaft 26 is closed and the sole member 12 is disposed within the insulating shaft 26 such that the bottom side 14 of the sole member 12 is attached to the closed distal end of the insulating shaft 26.

In some embodiments, such as the embodiments depicted in FIGS. 3, 7, 8, and 9, the insulating shaft 26 contains at least one zipper 38. The at least one zipper 38 secures the equine therapy boot 10 around the horse's hoof 18 and leg 22 to form a reservoir 48 for holding treatment media. In some embodiments, the at least one zipper 38 extends from a bottom 40 of the insulating shaft 26 to a top 42 of the insulating shaft 26. In some embodiments, the at least one zipper 38 extends from a top 42 of the insulating shaft 26 to a bottom 40 of the insulating shaft 26. In some embodiments, the at least one zipper 38 is a two-way zipper configured to be closed or opened in opposite directions at the same time. In some embodiments, the at least one zipper 38 is waterproof. Additionally, the at least one zipper 38 can also function as a drain to discharge the treatment media before removing the equine therapy boot 10 or refreshing the media for additional therapy. In some embodiments, the equine therapy boot 10 has a drain near the bottom 40 of the insulating shaft 26 to allow for easy draining of the media in the reservoir without removing the equine therapy boot 10 from the horse or releasing any treatment solids. Additionally, the at least one zipper 38 or drain can enable the user to make the equine therapy boot 10 leak treatment media continuously such that the reservoir 48 can be replenished intermittently.

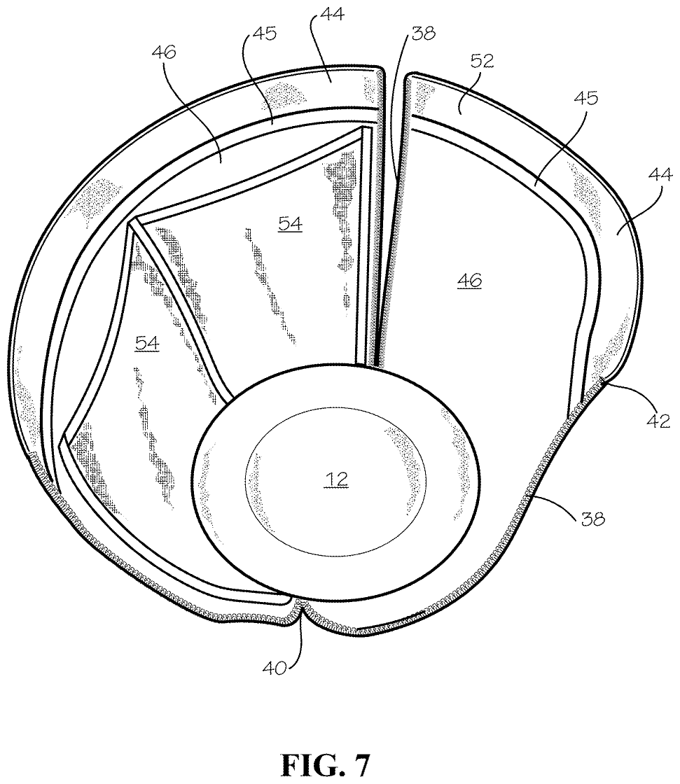

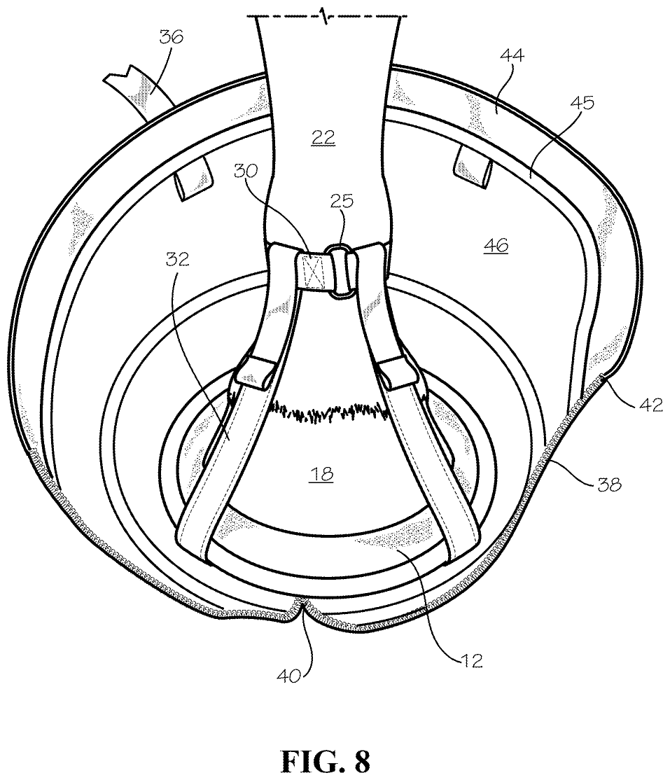

In some embodiments, such as the embodiments depicted in FIGS. 7 and 8, the insulating shaft 26 further includes an external layer 44, an insulating layer 45, and an internal layer 46. In some embodiments, the internal layer 46 and the external layer 44 are attached such that the insulating layer 45 is disposed between the external layer 44 and the internal layer 46. In some embodiments, the internal layer 46 is a waterproof material configured to prevent the treatment media contained in the reservoir 48 from leaking out of the equine therapy boot 10. In some embodiments, the insulating layer 45 is an insulating material that reduces the transmission of heat such that the temperature of the treatment media in the reservoir 48 is maintained for a period of time. In some embodiments, the insulating layer 45 is designed to retain the structure of the equine therapy boot 10 such that the equine therapy boot 10 is substantially non-deformable. In some embodiments, the external layer 44 is made of a protective material, such as Kevlar, designed to withstand wear and tear of the horse wearing the equine therapy boot 10. In some embodiments, the external layer 44 is made of a stiff material that provides substantially non-deformable structure to the equine therapy boot 10. In these embodiments, when the equine therapy boot 10 is fitted to the horse's leg and the at least one zipper 38 is closed, a reservoir 48 forms between the horse's hoof 18 and leg 22 and the internal layer 46. This configuration provides for direct topical treatment of treatment media to the horse's hoof 18 and leg 22. In these embodiments, the insulating shaft 26 preserves the temperature of warm or cold treatment media, thus reducing the amount of times the horse needs to be attended to throughout the treatment period.

In some embodiments, a mesh panel 54 is attached to the internal layer 46 to substantially form a pocket wherein ice, ice packs, or other treatment solids can be uniformly secured within the insulating shaft 22. The mesh panel 54 additionally provides a buffer between the treatment solids and the horse's limb, such that the horse is protected from injury such as burning or frost bite.

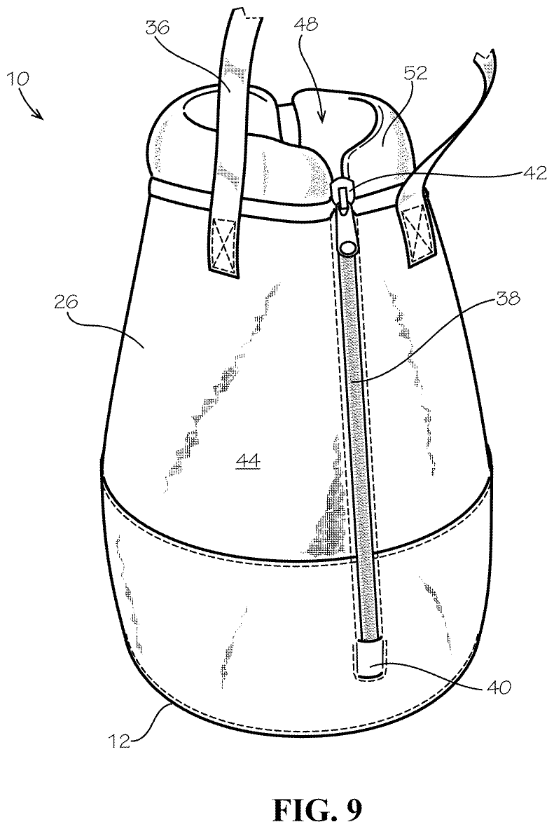

In some embodiments, the insulating shaft 26 further includes a top portion 52 wherein the top portion 52 substantially tapers around the horse's leg 22. In some embodiments the top portion 52 substantially tapers outward around the horse's leg 22. In other embodiments, such as the embodiment depicted in FIG. 9, the top portion 52 tapers inwards around the horse's leg 22. In some embodiments, the tapering configuration helps physically cover the reservoir 48 to prevent spillage of the treatment media and helps prevent debris from entering the reservoir 48 while providing additional insulation to the insulating shaft 26. In some embodiments, the top portion 52 of the equine therapy boot 10 is an adjustable cover made from deformable, non-chafing material that can be fastened by a removeable method such as hook and loop tape that keeps the top portion 52 closed around the horse's leg 22. In some embodiments, the top portion 52 is made from a deformable, non-chafing material such as fleece.

Methods for treating a diseased or injured horse's hoof and leg are disclosed herein. Methods of treatment include the step of providing an equine therapy boot 10, the equine therapy boot 10 comprising a sole member 12 having a bottom side 14 for contacting the ground and a top side 16 for contacting at least a portion of the sole of the horse's hoof 18, an internal securing mechanism 20 for securing the horse's leg 22 within the boot such that the portion of the sole of the horse's hoof 18 is in contact with the top side 16 of the sole member 12, the internal securing mechanism 20 further comprising a first plurality of straps 24 for attaching to a portion of the horse's leg 22 proximal to the horse's hoof 18, and an insulating shaft 26 wherein the insulating shaft 26 extends upwards from the sole member 12 wherein the insulating shaft 26 is configured to retain media. The method further includes fitting the equine therapy boot 10 to the horse. In some methods, the method includes administering media to the insulating shaft 26 of the equine therapy boot 10. In some methods, the method includes maintaining the equine therapy boot 10 in its position with the at least a portion of the sole of the horse's hoof 18 in contact with the top side 16 of the sole member 12 for a period of time.

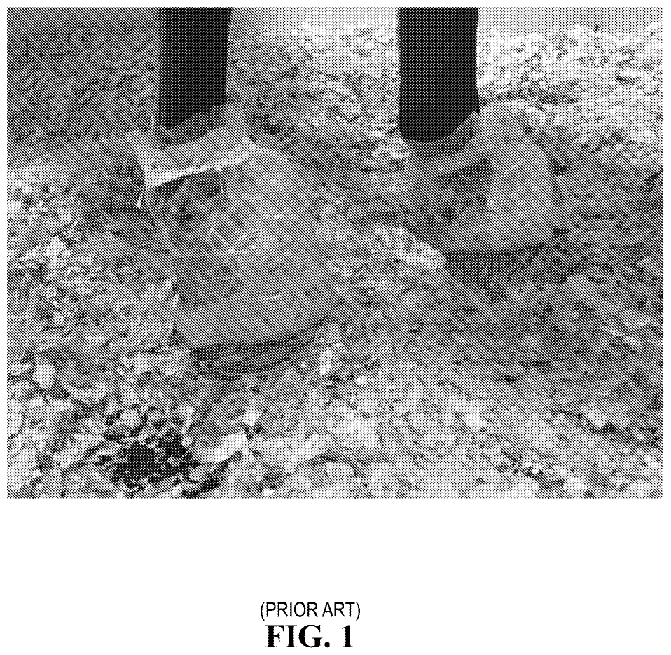

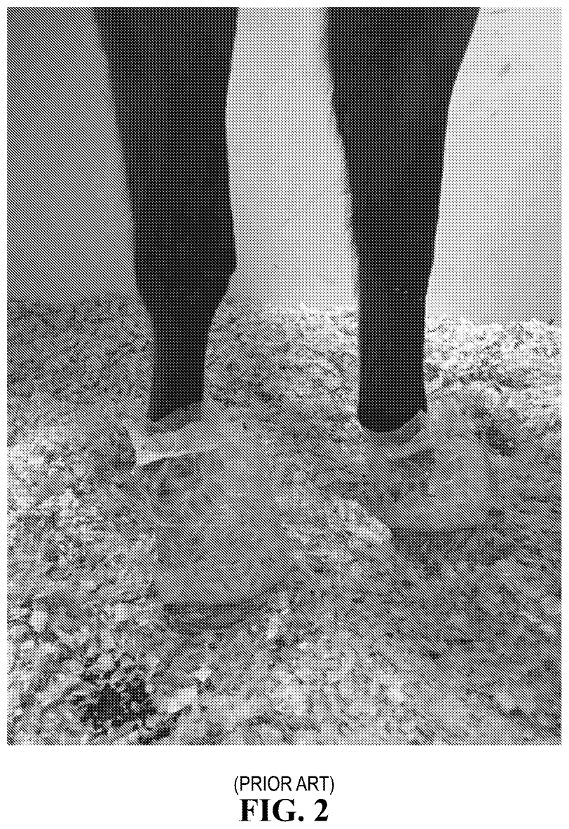

The method of treatment further includes maintaining the equine therapy boot 10 in its position with the at least a portion of the sole of the horse's hoof 18 in contact with the top side 16 of the sole member 12 for a period of time greater than 2 hours. The ice sleeve therapy method depicted in prior art FIGS. 1 and 2 illustrates the inefficiencies associated with attaching the ice sleeves to the horse's hoof and leg. For example, the horse often punctures the ice sleeves as the horse moves about, causing the ice sleeves to drain before the treatment period has concluded. Additionally, the ice contained in the uninsulated ice sleeves often melts before the treatment period has concluded decreasing the efficacy of the treatment. The combination of melting ice and difference between the temperature in the ice sleeves and the external environment causes sweat to develop on the external surface of the ice sleeves resulting in the adhesive tape detaching from the ice sleeves. This detachment contributes to the horse walking out of, or otherwise detaching itself from the treatment method. Accordingly, the caregiver must consistently monitor the horse and refresh the ice sleeves at least every two hours. Beneficially, the disclosed method can further include maintaining the equine therapy boot 10 in its position with the at least a portion of the sole of the horse's hoof 18 in contact with the top side 16 of the sole member 12 for a period of time of 4, 6, 8, 10, 12, 24, 48, 72 hours, or therebetween depending on the disease or injury and the advised treatment plan.

In some treatment methods, the administered media is cooler than the temperature of the horse's hoof 18. For example, inflammation of the laminae (laminitis) can often be prevented or abated by surrounding the horse's hoof 18 with cold water treatment for a period of time up to 72 hours. Additionally, physical injury and stress associated with extreme exercise can be effectively treated with cold water treatment to diffuse swelling. In other treatment methods, the administered media is warmer than the temperature of the horse's hoof 18. For example, after draining a hoof abscess, the horse's hoof can be soaked in warm treatment media, such as a combination of salt and warm water, before being dried and bandaged. Similarly, corns, or bruises between the frog and the hoof can be treated by warm foot baths and poultices.

What is claimed is:

1. An equine therapy boot comprising:
a sole member having a bottom side for contacting the ground and a top side for contact with at least a portion of the sole of a horse's hoof;
an internal securing mechanism for securing the horse's leg within the boot such that the portion of the sole of the horse's hoof is in contact with the top side of the sole member, the internal securing mechanism further comprising a first plurality of straps for attaching to a portion of the horse's leg proximal to the horse's hoof; and
an insulating shaft wherein the insulating shaft extends upward from the sole member wherein the insulating shaft is configured to retain media.

2. The equine therapy boot of claim 1 wherein the insulating shaft is secured to the horse's leg by a second plurality of straps at a point proximal to the first plurality of straps.

3. The equine therapy boot of claim 1 wherein the first plurality of straps further comprises a first garter strap configured to extend around the horse's pastern and at least one suspending strap that extends from the sole member to the first garter strap.

4. The equine therapy boot of claim 2 wherein the second plurality of straps further comprises a second garter strap configured to extend around the horse's leg and at least one suspending strap that extends from the insulating shaft to the second garter strap.

5. The equine therapy boot of claim 1 wherein the insulating shaft is attached to the sole member.

6. The equine therapy boot of claim 1 wherein the distal end of the insulating shaft is closed and the sole member is disposed within the insulating shaft such that the bottom side of the sole member is attached to the closed distal end of the insulating shaft.

7. The equine therapy boot of claim 1 wherein the insulating shaft contains at least one zipper extending from a bottom of the insulating shaft to a top of the insulating shaft.

8. The equine therapy boot of claim 7 wherein the at least one zipper is a two-way zipper.

9. The equine therapy boot of claim 1 wherein the insulating shaft further comprises an external layer, an insulating layer, and an internal layer, wherein the external layer, the insulating layer, and the internal layer form a reservoir for retaining media.

10. The equine therapy boot of claim 9 wherein the internal layer is attached to the external layer.

11. The equine therapy boot of claim 9 wherein the external layer comprises a protective material.

12. The equine therapy boot of claim 9 wherein the insulating layer comprises an insulating material that reduces the transmission of heat.

13. The equine therapy boot of claim 9 wherein the internal layer comprises a waterproof material.

14. The equine therapy boot of claim 9 further comprising a mesh panel attached to the internal layer.

15. The equine therapy boot of claim 1 wherein the insulating shaft further comprises a top portion wherein the top portion substantially tapers around the horse's leg.

16. The equine therapy boot of claim 1 wherein the sole member is a cushioning pad.

17. A method of treating a diseased or injured horse's hoof and leg comprising;
providing an equine therapy boot, the equine therapy boot comprising a sole member having a bottom side for contacting the ground and a top side for contacting at least a portion of the sole of the horse's hoof, an internal securing mechanism for securing the horse's leg within the boot such that the portion of the sole of the horse's hoof is in contact with the top side of the sole member, the internal securing mechanism further comprising a first plurality of straps for attaching to a portion of the horse's leg proximal to the horse's hoof, and an insulating shaft wherein the insulating shaft extends upwards from the sole member wherein the insulating shaft is configured to retain media;
fitting the equine therapy boot to the horse;
administering media to the insulating shaft of the equine therapy boot; and
maintaining the equine therapy boot in its position with the at least a portion of the sole of the horse's hoof in contact with the top side of the sole member for a period of time.

18. The method of claim 17 wherein the equine therapy boot is maintained in its position with the at least a portion of the sole of the horse's hoof in contact with the top side of the sole member for a period of time greater than 2 hours.

19. The method of claim 18 wherein the equine therapy boot is maintained in its position with the at least a portion of the sole of the horse's hoof in contact with the top side of the sole member for a period of time of 4, 6, 8, 10, 12, 24, 48, 72 hours, or therebetween.

20. The method of claim 17, wherein the media is cooler than the temperature of the horse's hoof.

21. The method of claim 17, wherein the media is warmer than the temperature of the horse's hoof.
